(19) United States
(12) Patent Application Publication
(10) Pub. No.: US 2016/0008576 A1
(43) Pub. Date: Jan. 14, 2016

(54) PERCUTANEOUS PORT GUARD DRESSING

(71) Applicant: LIG SCIENCES, INC., Export, PA (US)

(72) Inventors: Sang Beom Lee, Harrison City, PA (US); Kevin Smith, Latrobe, PA (US)

(21) Appl. No.: 14/796,205

(22) Filed: Jul. 10, 2015

Related U.S. Application Data
(60) Provisional application No. 62/024,245, filed on Jul. 14, 2014.

(51) Int. Cl. A61M 25/02

(52) U.S. Cl. CPC A61M 25/02; A61M 2025/0246; A61M 2025/0253; A61M 2207/00

(57) ABSTRACT

A port guard dressing for use in connection with skin of a patient and through which a percutaneous line can pass includes an upper component, a lower component adapted to contact the skin, and at least one intermediate component positioned between the upper component and the lower component. The at least one intermediate component is attached to a lower surface of the upper component and includes or is formed from a material that is more rigid than the upper component. The material of the of the upper component intermediate component may also be more rigid than the lower component.

### CROSS-REFERENCE TO RELATED APPLICATIONS

This application claims benefit of U.S. Provisional Patent Application Ser. No. 62/024,245, filed Jul. 14, 2014, the disclosure of which is incorporated herein by reference.

### GOVERNMENTAL INTEREST

This invention was made with government support under grant nos. 2R44HL106929-02 and R44HL106929-03 awarded by the National Heart, Lung and Blood Institute of the National Institutes of Health. The government has certain rights in this invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

As part of the advancements of medicine and various medical treatments and procedures, there are an increased number of percutaneous ports and uses of percutaneous ports. Examples of percutaneous ports or lines include, but are not limited to, drivelines for Ventricular Assist Devices (VAD), feeding tubes, drain tubes, dialysis tubes, and catheters (sometime referred to herein collectively as “percutaneous lines”). These percutaneous lines may be in place for extended periods of time and often remain in use even after a patient leaves the hospital and returns to a home environment. The dressings utilized around the percutaneous opening are often makeshift, not very secure and not easy changed. Moreover, such dressings cannot be easily worn while bathing or showering.

Furthermore, such dressings are a source of skin irritations (from, for example, skin-adhering dressing adhesive area(s)) and do not adequately protect against various types of infections. Infections have become a major concern for hospital patients and those that may be in recovery from surgery. Patients having long-term percutaneous ports are particularly prone to infection.

VADs used today need to be powered by an external power source, usually electrical or pneumatic, via a driveline that contains electrical wire and/or tubing that is connected to an implanted blood pumping device and exits the body via a percutaneous hole in the abdomen. The exit point is a key concern, particularly as an entry point for infection causing microorganisms. Such infections are often sufficiently serious to cause extended hospital stays and even death. It has been stated that a third of the mortalities of patients having a VAD are directly related to infections beginning at the entry site of the driveline.

VAD driveline sheaths may include a velour type of cuff material at the entry site that facilitates skin in-growth into the velour, thus creating a seal of the opening. However, in-growth can take time (for example, on the order of weeks), requires complete immobilization of the driveline while skin in-growth is occurring, and can sustain damage by any accidental pulling/pushing of the driveline causing it to be torn away from the in-grown skin.

### SUMMARY

In one aspect, a port guard dressing for use in connection with skin of a patient and through which a percutaneous line can pass includes an upper component, a lower component adapted to contact the skin, and at least one intermediate component positioned between the upper component and the lower component. The at least one intermediate component is attached to a lower surface of the upper component and includes or is formed from a material that is more rigid than the upper component. The material of the intermediate component may also be more rigid than the lower component.

The port guard dressing may further include at least one absorbent component positioned below the at least one intermediate component and above the lower component. The absorbent component may, for example, include a hydroscopic material. In a number of embodiments, the absorbent component includes at least one of absorptive fibers, an absorptive powder or an absorptive gel.

The lower component may, for example, include an antimicrobial material. The antimicrobial material may, for example, include at least one of silver, a silver compound, iodine, an iodine compound, tetracycline, an antimicrobial peptide, or a quaternary ammonium compound.

1111 In a number of embodiments, the upper component includes a passage through which the percutaneous line can pass and a slit in connection with the passage of the upper component and extending to a perimeter of the upper component. The first intermediate component may, for example, include a passage through which a percutaneous line can pass and a slit in connection with the passage of the intermediate component and extending to a perimeter of the intermediate component. The lower component may, for example, include a passage through which a percutaneous line can pass and a slit in connection with the passage of the lower component and extending to a perimeter of the lower component. The passage of the upper component, the passage of the intermediate component and the passage of the lower component may, for example, be in general alignment. In a number of embodiments, the slit of the intermediate component and the slit of the lower component are in general alignment and are offset from the slit of the upper component. In a number of other embodiments, all component slits are in general alignment, with generally no offset therebetween.

As described above, the port guard dressing may further include at least one absorbent component positioned below the at least one intermediate component and above the lower component. The absorbent component may, for example, include a passage through which a percutaneous line can pass and a slit in connection with the passage of the absorbent component and extending to a perimeter of the absorbent component. The passage of the absorbent component may, for example, be in general alignment with the passage of the upper component, the passage of the intermediate component and the passage of the lower component. In a number of embodiments, the slit of the absorbent component is in general alignment with the slit of the intermediate component and the slit of the lower component, while be offset from the slit of the upper component.

The upper component may, for example, include a first section including an adhesive on a lower surface thereof adjacent the slit of the upper component via which the upper component is adapted to be adhered to at least one of the intermediate component and the lower component after the percutaneous line is positioned to pass through the passage of the upper component, the passage of the intermediate component and the passage of the lower component via the slit of the intermediate component and the slit of the lower component. The first section may, for example, be covered with a first section of a removable layer.

In a number of embodiments, the upper component is adhered to at least one of the intermediate component and the lower component via an adhesive positioned on a lower surface of the upper component outside of the first section of the upper component during fabrication of the port guard dressing. The lower component may, for example, have an area larger than an area of the intermediate component, and the upper component may be adhered to the lower component during fabrication of the port guard dressing. In a number of embodiments, an area of the upper component is larger than an area of the intermediate component and an area of the lower component, and the upper component includes a second section including an adhesive on the lower surface thereof via which the port guard dressing is adapted to be attached to the skin of the patient. The second section may, for example, extend around a perimeter of the upper component. The second section may, for example, be covered with a second section of a removable layer. In a number of embodiments, the second section is formed to have areas without adhesive which are adapted to decrease skin irritation. The second section may, for example, include a plurality of openings formed therein which are adapted to decrease skin irritation. The openings may, for example, extend from a perimeter of the second section inward toward the passage of the first component.

In a number of embodiments, the upper component includes a plurality of holes therein and the intermediate component comprises a plurality of holes therein. The plurality of holes of the upper component may, for example, be generally aligned with the plurality of holes of the intermediate component. The plurality of holes of the upper component may, for example, have a diameter in the range of 0.06 to 0.5 inches, and the plurality of holes of the intermediate component may, for example, have a diameter in the range of 0.06 to 0.5 inches.

In a number of embodiments, the intermediate component includes an adhesive layer on a lower surface thereof.

In a number of embodiments, the lower component includes a layer of material adapted to contact the skin and a generally axially oriented cylindrical extending member which extends from the layer of material of the lower component through the passage of the intermediate component and the passage of the upper component. The extending member may, for example, include an axially extending slit therein in general alignment with the slit of the intermediate component and the slit of the lower component through which the percutaneous line may pass to be placed within the extending member. In a number of embodiments, the axially oriented cylindrical extending member is formed from a rectangular piece of material including tabs along the bottom thereof. The rectangular piece of material is rolled to form the extending member and inserted into the passage of the bottom layer, with the axially extending slit of the extending member and slit of the lower component in alignment. The tabs may be bent radially outward and attached to the lower component.

The port guard dressing may, for example, include a strip of material which is adapted to be wrapped around the extending member and the percutaneous line. The strip of material may, for example, include an adhesive or is self-adhering. The strip of material may, for example, be attached to the extending member at one end thereof. In a number of embodiments, the strip of material has an axial height in the range of 0.5 to 2.0 inches and a length in the range of 1.5 to 4.0 inches.

In another aspect, a port guard dressing for use in connection with skin of a patient and through which a percutaneous line can pass includes an upper component, a lower component adapted to contact the skin, and at least one intermediate component positioned between the upper component and the lower component. The upper component includes a passage through which the percutaneous line can pass and a slit in connection with the passage of the upper component and extending to a perimeter of the upper component. The at least one intermediate component includes a passage through which a percutaneous line can pass and a slit in connection with the passage of the intermediate component and extending to a perimeter of the intermediate component. The lower component includes a passage through which a percutaneous line can pass and a slit in connection with the passage of the lower component and extending to a perimeter of the lower component. The passage of the upper component, the passage of the intermediate component and the passage of the lower component are in general alignment. The slit of the intermediate component and the slit of the lower component are in general alignment, and the slit of the upper component is offset from the slit of the intermediate component and the slit of the lower component.

The upper component may, for example, include a first section including an adhesive on a lower surface thereof adjacent the slit of the upper component via which the upper component is adapted to be adhered to at least one of the intermediate component and the lower component after the percutaneous line is positioned to pass through the passage of the upper component, the passage of the intermediate component and the passage of the lower component via the slit of the intermediate component and the slit of the lower component. The first section may, for example, be covered with a first section of a removable layer.

The upper component may, for example, be adhered to at least one of the intermediate component and the lower component via an adhesive positioned on a lower surface of the upper component outside of the first section of the upper component during fabrication of the port guard dressing. The lower component may, for example, have an area larger than an area of the intermediate component, and the upper component may be adhered to each of the intermediate component and the lower component during fabrication of the port guard dressing. In a number of embodiments, an area of the upper component is larger than an area of the intermediate component and an area of the lower component and the upper component includes a second section including an adhesive on the lower surface thereof via which the port guard dressing is adapted to be attached to the skin of the patient. The second section extends around a perimeter of the upper component. The second section may, for example, be covered with a second section of a removable layer.

The second section may, for example, be formed to have areas without adhesive which are adapted to decrease skin irritation. The second section may, for example, include a plurality of openings formed therein which are adapted to decrease skin irritation. The openings may, for example, extend from a perimeter of the second section inward toward the passage of the first component.

In another aspect, a method of fabricating a port guard dressing for use in connection with skin of a patient and through which a percutaneous line can pass includes providing an upper component, providing a lower component adapted to contact the skin, providing at least one intermediate component positioned between the upper component and the lower component, attaching the at least one intermediate component to a lower surface of the upper component, the at least one intermediate component including a material that is more rigid than the upper component. The material of the intermediate component may also be more rigid than the lower component.

In another aspect, a method of fabricating a port guard dressing for use in connection with skin of a patient and through which a percutaneous line can pass includes providing an upper component, providing a lower component adapted to contact the skin, providing at least one intermediate component positioned between the upper component and the lower component, the upper component including a passage through which the percutaneous line can pass and a slit in connection with the passage of the upper component and extending to a perimeter of the upper component, the at least one intermediate component including a passage through which a percutaneous line can pass and a slit in connection with the passage of the intermediate component and extending to a perimeter of the intermediate component, and the lower component including a passage through which a percutaneous line can pass and a slit in connection with the passage of the lower component and extending to a perimeter of the lower component, the passage of the upper component, the passage of the intermediate component and the passage of the lower component being in general alignment, the slit of the intermediate component and the slit of the lower component being in general alignment, and the slit of the upper component being offset from the slit of the intermediate component and the slit of the lower component.

In a further aspect, a method of securing a percutaneous line includes use of a port guard dressing as set forth herein.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a perspective view of a port guard dressing hereof in an assembled state.

FIG. 1B illustrates another perspective view of the port guard dressing of FIG. 1A including a strip of material adapted to be wrapped around and secure a percutaneous line.

FIG. 2 illustrates a perspective view of the port guard dressing of FIG. 1A in a disassembled or exploded state.

FIG. 3 illustrates a bottom view of an embodiment of a first or upper component of the port guard dressing of FIG. 1A.

FIG. 4 illustrates a bottom view of an embodiment of a second, intermediate component of the port guard dressing of FIG. 1A which provides stiffness and/or mechanical strength to the port guard dressing.

FIG. 5 illustrated a bottom view of an embodiment of a third, intermediate component of the port guard dressing of FIG. 1A which provides an absorbent layer.

FIG. 6 illustrates an embodiment of a fourth, lower or bottom component of the port guard dressing of FIG. 1A which contacts the skin of the patient in the area surrounding the percutaneous line and opening.

FIG. 7A illustrates a perspective view of the fourth, lower or bottom component of the port guard dressing of FIG. 1A.

FIG. 7B illustrates a perspective view of a methodology for fabricating the fourth, lower or bottom component of the port guard dressing of FIG. 1A.

FIG. 8A illustrates a top view of another embodiment of a bandage or dressing including areas in which an adhesive is absent in the form of openings or narrow slits in the bandage or dressing which reduce or minimize skin irritation resulting from the mismatch of elasticity of skin and the component or layer including the adhesive used to adhere the bandage or dressing to the skin.

FIG. 8B illustrates a top view of another embodiment of a bandage or dressing including areas in which an adhesive is absent in the form of openings or narrow slits in the bandage or dressing which reduce or minimize skin irritation resulting from the mismatch of elasticity of skin and the component or layer including the adhesive used to adhere the bandage or dressing to the skin.

FIG. 9 illustrates a top view of another embodiment of a bandage or dressing including areas in which an adhesive is absent in the form of openings larger than the relatively narrow slits of FIGS. 8A and 8B in the bandage or dressing which reduce or minimize skin irritation resulting from the mismatch of elasticity of skin and the component or layer including the adhesive used to adhere the bandage or dressing to the skin.

FIG. 10 illustrates a top view of another embodiment of a bandage or dressing including areas of a bottom surface of a component of the bandage or dressing in which an adhesive is absent to reduce or minimize skin irritation resulting from the mismatch of elasticity of skin and the component or layer including the adhesive used to adhere the bandage or dressing to the skin.

FIG. 11 illustrates a top view of another embodiment of a bandage or dressing including areas of a bottom surface of a component of the bandage or dressing in which an adhesive is absent to reduce or minimize skin irritation resulting from the mismatch of elasticity of skin and the component or layer including the adhesive used to adhere the bandage or dressing to the skin.

FIG. 12A illustrates a percutaneous line such as a PICC line in place in connection with a patient.

FIG. 12B illustrates placement of a port guard dressing hereof in operative connection with the percutaneous line.

FIG. 12C illustrates removal of a peel away layer or section to effect closure of a portion of the upper component of the port guard dressing with one or more lower components in the vicinity of a slit through the upper component which enable connection of the percutaneous line with a central passage through the port guard dressing.

FIG. 12D illustrates the port guard dressing after removal of the peel away layer of FIG. 12C to expose underlying adhesive.

FIG. 12E illustrates connection of the portion of the upper component with the lower component(s).

FIG. 12F illustrates removal of another peel away layer or section to expose adhesive to connect the port guard dressing with the patient's skin.

FIG. 12G illustrates removal of the peel away layer or section to expose adhesive to connect the port guard dressing with the patient's skin

FIG. 12H illustrates contact of the port guard dressing with the patient's skin.

FIG. 12I illustrates application of pressure to the port guard dressing to form an adhering bonds between the port guard dressing and the patient's skin.

FIG. 12J illustrates removal of a peel away layer or section from a length of material which forms an extending, tubular member of the port guard dressing through which the percutaneous line passes.

FIG. 12K illustrates encompassing of the percutaneous line by the extending, tubular member.

FIG. 12L illustrates the finished port guard dressing in connection with the patient and the percutaneous line.

FIG. 13A illustrates another perspective view of the port guard dressing of FIG. 1A illustrating that a member encompassing a length of a tube or line passing through the port guard dressing can be bent to various angles.

FIG. 13B illustrated the port guard dressing of FIG. 1A wherein the member encompassing a length of a tube or line (for example, a peripherally inserted central catheter (PICC) or other catheter, tube or line) is bent so that the tube or line can be passed through a securement attached to the patient's skin.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a layer” includes a plurality of such layers and equivalents thereof known to those skilled in the art, and so forth, and reference to “the layer” is a reference to one or more such layers and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

In addition, minimizing or eliminating the risk of infection, a port guard dressing for use in connection with a percutaneous line such as a VAD driveline is required to be in place for many days, weeks, if not for months or years. Such a port guard dressing should not only be able to be in place for extended periods of time, but should also be easily changed and maintained by personnel with minimal or no medical training. While providing a physical securement of the percutaneous line, the port guard dressing should also cause minimal, if any, discomfort from port site infections and skin irritations caused from the skin-adhesive portions of the dressing. In general, a port guard dressing should provide peace of mind to the patient and enable the patient to lead a life as close as possible to a “normal”, unencumbered way of life (including, for example, participation in the usual daily routines of bathing, showering, exercise, work, etc.).

Port guard dressings hereof greatly alleviate problems at the percutaneous entry site for percutaneous lines and/or other port applications. For example, port guard dressings hereof provide an actual physical barrier, which can be treated with an antibacterial coating, for preventing the passage of unwanted micro-organisms through the percutaneous opening. Further, port guard dressings hereof firmly secure the percutaneous line to the body to prevent unwanted movement relative to the body with respect to the driveline. One type of unwanted percutaneous line movement is inward and outward motion from the body. This type of motion not only poses a risk of damage to the percutaneous line and associated devices, but also may harm internal tissue and organs. The inward/outward movement of the percutaneous line also enables the dangerous entry of harmful, and in some cases lethal, infection causing micro-organisms, which may initially reside on the percutaneous line at the immediate entry site and will end up inside of the body when the percutaneous line is pushed into the body. Another type of unwanted motion, side-to-side motion of the percutaneous line, can cause an enlargement of the percutaneous entry hole, further increasing the risk of infection. In addition to the above mentioned benefits, the port guard dressings hereof enhance peace of mind of the patient by ensuring that the port line is secure with the entry point being hidden under the dressing and that the entry point is secured, sealed and protected from the outside elements.

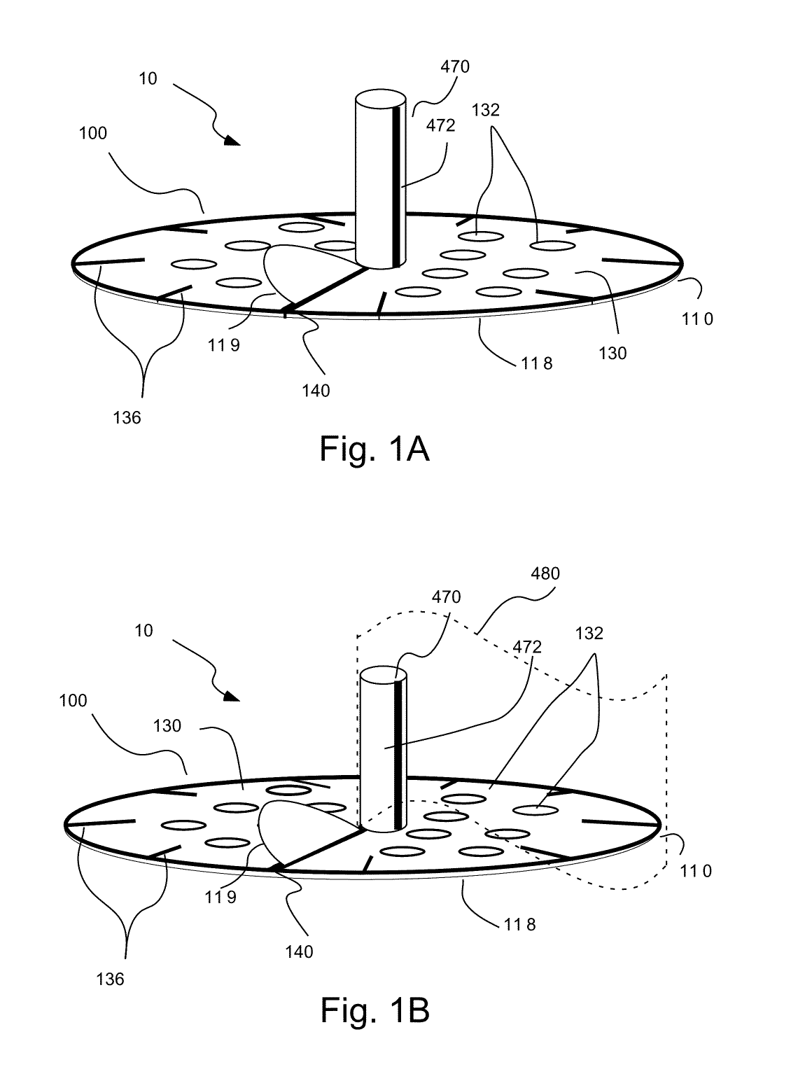

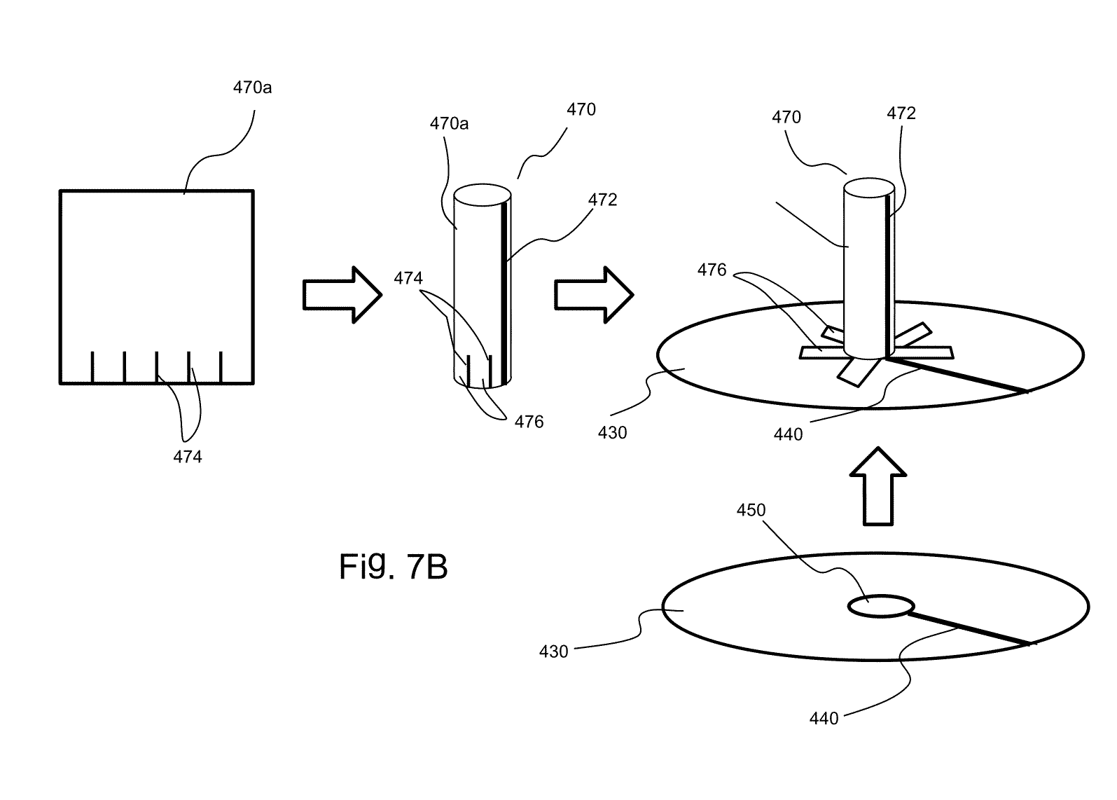

In general, port guard dressings hereof include an upper component having an upper surface which is in contact with the surrounding environment and a lower component having a lower surface which is in contact with the skin of a patient when the port guard dressing is in use in connection with a patient. Terms such as up, down, upper, lower, above, below and like terms refer to an orientation of the port guard dressing as worn by the patient wherein upper is away from the patients skin and lower is toward the patients skin. The term radial and like terms refers to a direction generally perpendicular to an axis defined by an opening or passage in the port guard dressing through which a percutaneous port or line passes. The port guard dressings may be, but need not be, formed circularly and/or generally symmetrically about the opening or passage. The port guard dressing hereof also include one or more intermediate components positioned intermediate or between the upper component and the lower component. In the embodiment set forth in FIGS. 1A through 7C port guard dressing or bandage 10 includes four components.

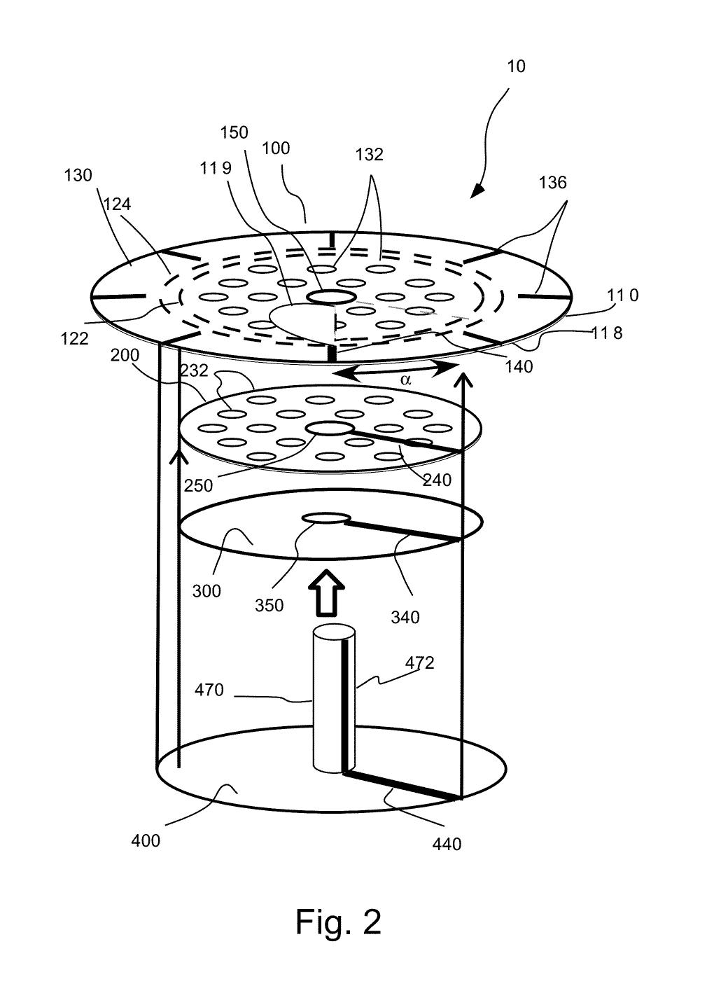

FIGS. 1A and 1B illustrate a port guard dressing or bandage 10 hereof in an assembled state, while FIG. 2 illustrated port guard dressing 10 in a disassembled or exploded state. Port guard dressing 10 is the end product of a number of components or layers (once again, four in the illustrated embodiment) which are oriented as illustrated in FIG. 2 and assembled/laminated together. The assembled port guard dressing 10 is sterilized and packaged for use. Port guard dressing 10 may, for example, be applied to a patient by a doctor, nurse, an aide or even a patient.

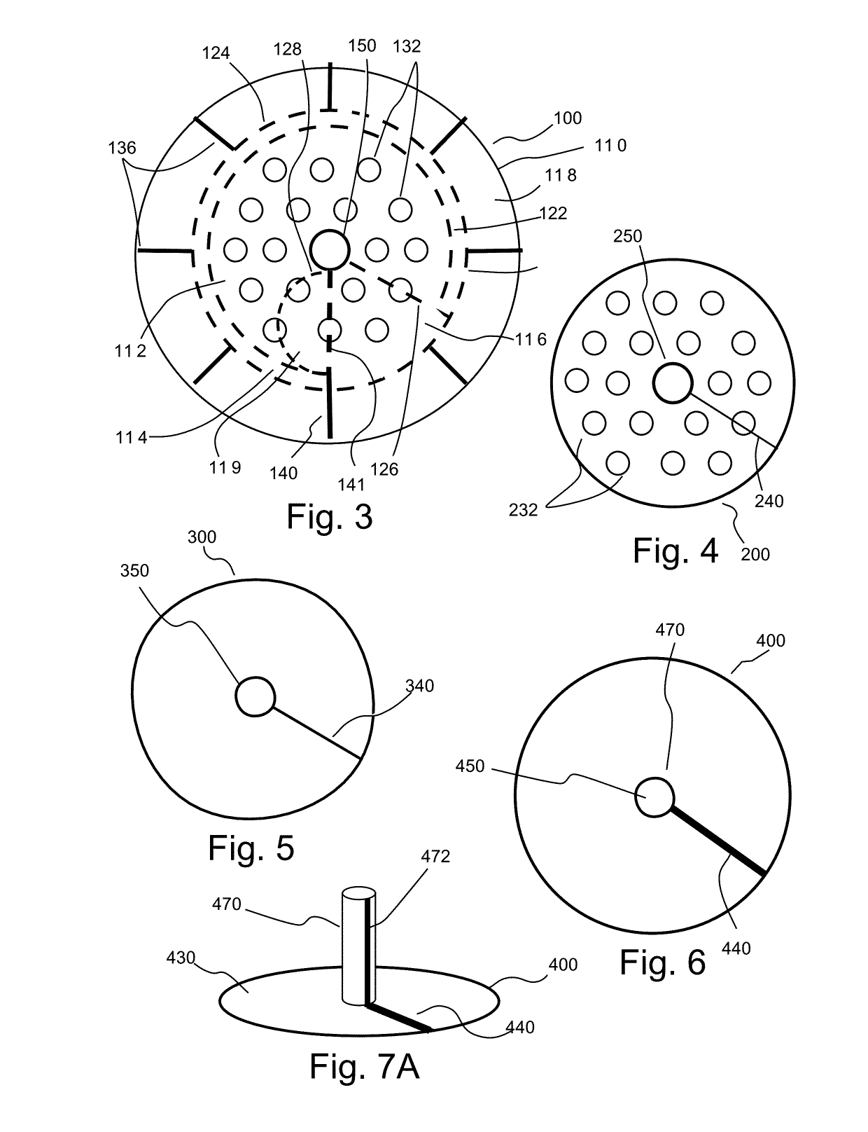

FIGS. 3 through 7 illustrate embodiments of components that are used to construct port guard dressing 10 of FIG. 1A. Although the representative example of port guard dressing 10 and various components thereof are illustrated as circular, other shapes (such as, for example, an oval, rectangle, square, or another symmetrical or irregular shape) are possible. As described further below, each component has one or more specific roles or functions in the overall functionality of the port guard dressing. In a number of embodiments, upon assembly, the components enable port guard dressing 10 to, for example: 1) have sufficient strength and stiffness around a passage for the tube/driveline to be firmly secured to the dressing and therefore the body: 2) effectively mend together the two section or ends of a dressing opening or slit by the overlap of a top layer slit by, for example, a top component or layer which is rotated in orientation with respect to the other components or layers; 3) inhibit the transport of harmful micro-organisms to the port site by physically sealing the area with the sterilized dressing that acts as a barrier to micro-organism (with the option of using an antibacterial agent); 4) absorb fluids (including, for example, absorbing body fluids or fluids such as sterilized water for activating antimicrobial agents); and 5) minimize skin irritation as compared to other dressings and/or bandages.

In a number of embodiments, one or more components of port guard dressing 10 include layers or films of materials with an adhesive on at least one side thereof. The adhesive may, for example, be covered by a peel away layer that can be removed during assembly (to bond adjacent layers) or upon application to patient. FIG. 3 shows a lower surface of a first, upper or outer layer or component 100 of port guard dressing 10 that includes a removable or peel away layer 110 connected to an upper or outer layer 130 that covers and protects an adhesive layer or layers (as known in the adhesive and bandage arts). In the illustrated embodiment, peel away layer 110 is divided into a number of sections. For example, cuts may be made in the peel away layer 110 of upper component 100 while it is connected to outer layer 130 to a depth that cuts only peel away layer 110. These cuts, which define sections 112, 114, 116 and 118 of peel way layer 110 are represented by dashed lines 122, 124 and 126 in FIG. 3.

FIGS. 2-7B illustrate the components of the dressing (shown individually in FIGS. 3-7B) and the alignment of the components relative each other as present in assembled port guard dressing 10. Outer layer 130 of first, upper component 100 is formed of a flexible dressing material (as known in the bandage arts). The material used for outer layer 130 may, for example, be a medical bandage material such as cloth material, a woven material, a non-woven material, or an elastomeric polymer material. Examples of suitable materials include polyamide, polyethylene, polyester, polyacrylate, and cotton. Outer layer 130 may, for example, have a flesh colored top or outer surface to blend in with the skin. The adhesive coating(s) on the lower surface of outer layer 130 is covered by peel away layer 110 as described above, which may be a wax-paper-type peel away layer as known in the adhesive arts, which when peeled away exposes the adhesive so that the dressing can adhere to the skin (in the radially outer circumferential area defined by dashed line 124 and underlying section 118 of peel away layer 110) or to second (intermediate) component 200 and fourth, lower or bottom component 400 (in the radially inward are defined by dashed circular line 124).

In FIG. 3, cuts completely through outer layer 130 and peel away layer 110 are shown as solid dark lines (for example, cuts or slits 136 and slit 140, which are discussed further below). A cut through only outer layer 130 and not upper peel away layer 110 is shown as a heavy dashed line 141 in FIG. 3 (that is, dashed line 141 forming a portion of slit 140). Cuts through only peel away layer 110 and not outer layer 130 are shown as the lighter or thinner dashed lines 122, 124, 126 and 128. The center circular hole or passage 150 and vent holes or passages 132 (illustrated in solid lines) are cut through both peel away layer 110, the adhesive layer and outer layer 130 of upper or first component 100. Generally central passage 150 provides for the passage of a percutaneous line therethrough.

Vent holes 132 may be provided in layer 130 of first or upper component 100 to, for example, observe any blood or bodily fluids that may emanate from the percutaneous opening in the patient and/or to add of water and/other fluids to activate a antimicrobial coating that may be imparted on a fabric layer 430 of component 400 (discussed further below) for infection control. Such an antimicrobial coating may, for example, include a silver-based material, an iodine-based material, tetracycline, antimicrobial peptides materials, quaternary ammonium compounds and/or other anti-microbial inhibiting agents.

Second component 200 is an intermediate component which, in the illustrated embodiment, is positioned adjacent and below first component 100 in assembled port guard dressing 10. Vent hole 232 in second component 200 align with vent holes 132 in first component 100. In a number of embodiment, holes 232 were cut with second component 200 and first component 100 adhered together. Second component 200, as illustrated in FIGS. 2 and 4 is flexible but is more rigid than first component 100 and the other components. Second component 200 provides strength and a little stiffness to port guard dressing 10 so as to be able to firmly secure the material holding a percutaneous line by, for example, distributing any perpendicular/side and parallel/up and down external forces across the area of port guard dressing 10 and the dressing adhesive. Second component 200 may, for example, be formed from a polymeric film such as polyester, polycarbonate, polypropylene, and polyethylene, each having one side with an adhesive covered with a peel away layer. In a number of embodiments, the material may be a clear stiff plastic 0.05 mm to 1.6 mm thick with an adhesive on one side covered by a peel away layer. Another function of second component 200 is to ensure that fourth, bottom component 400, which may have an antibacterial coating or treatment, contacts the skin evenly up to the percutaneous opening in the skin. Second component 200 assists in minimizing or eliminating wrinkles or openings that may otherwise develop between the skin and port guard dressing 10, which would undesirably allow a path for bacteria to travel to the percutaneous site.

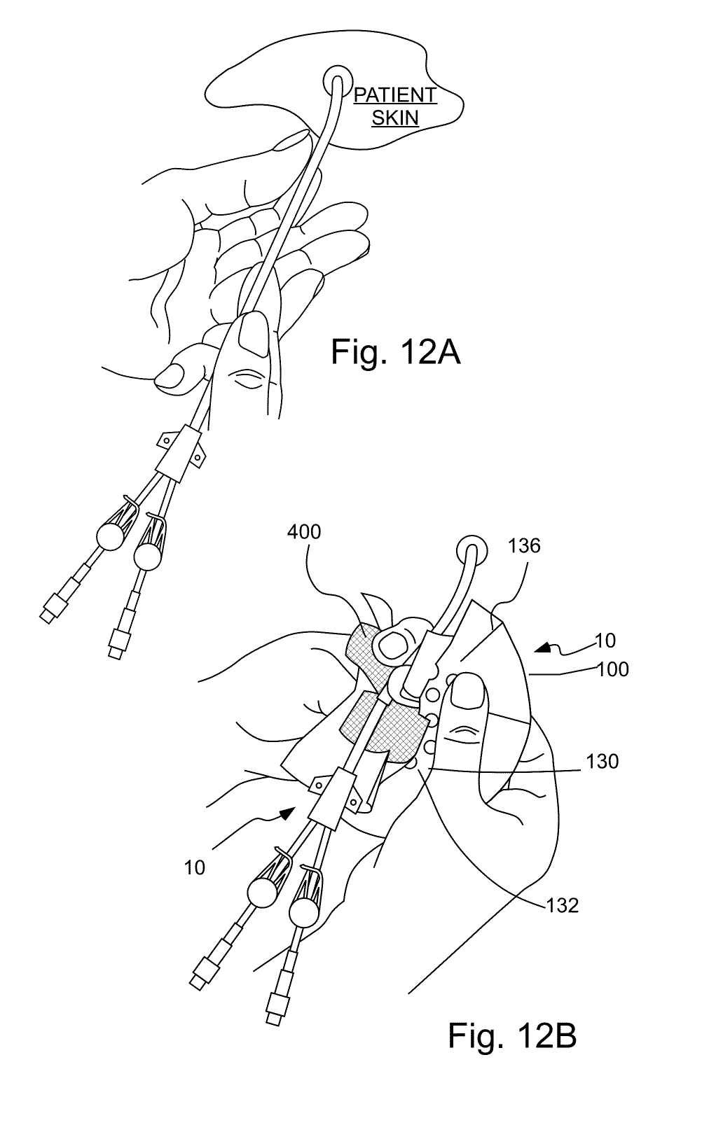

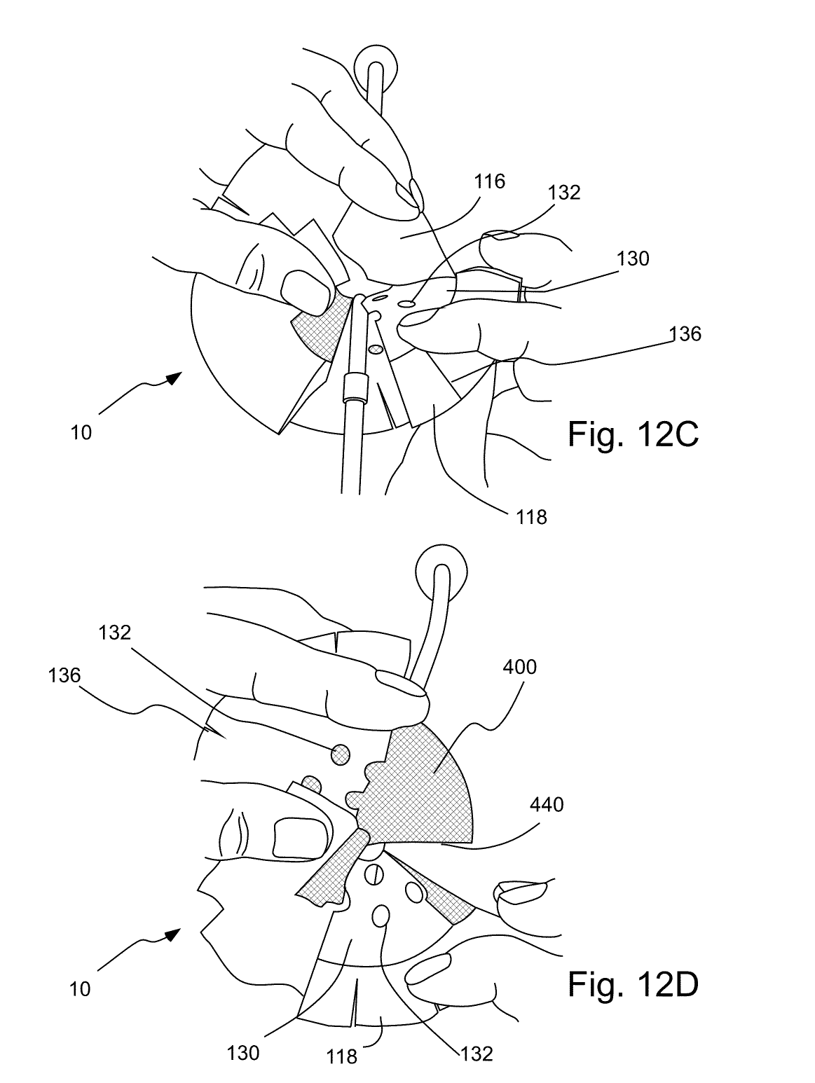

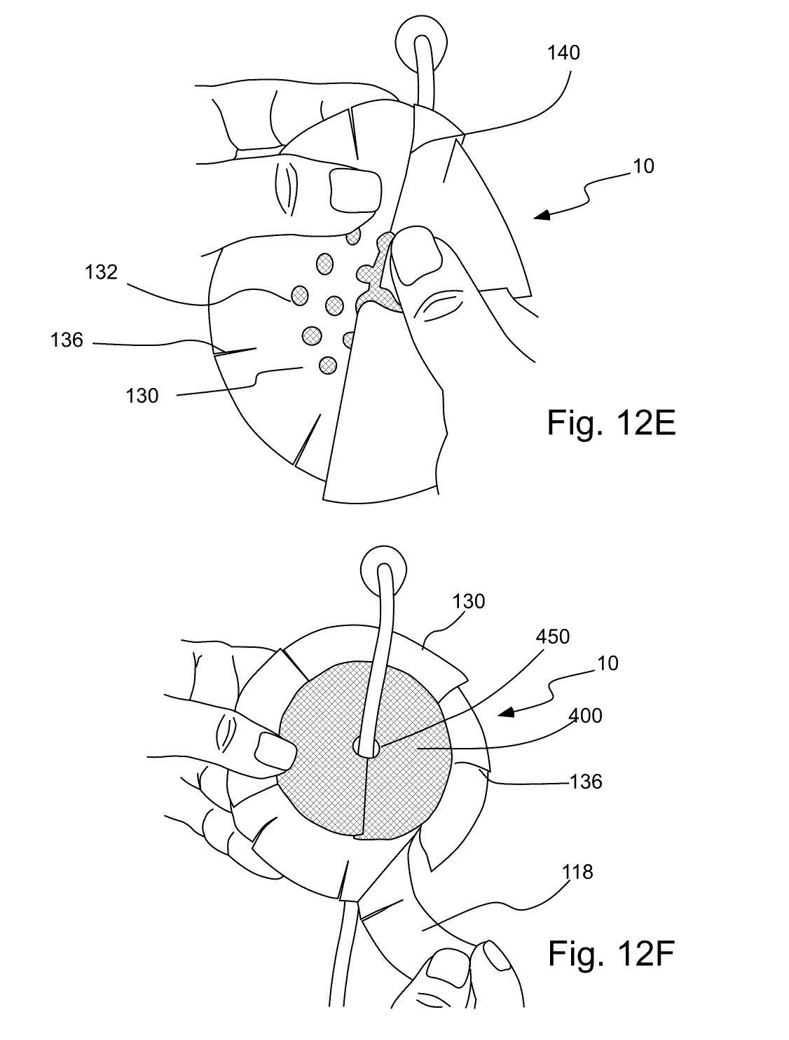

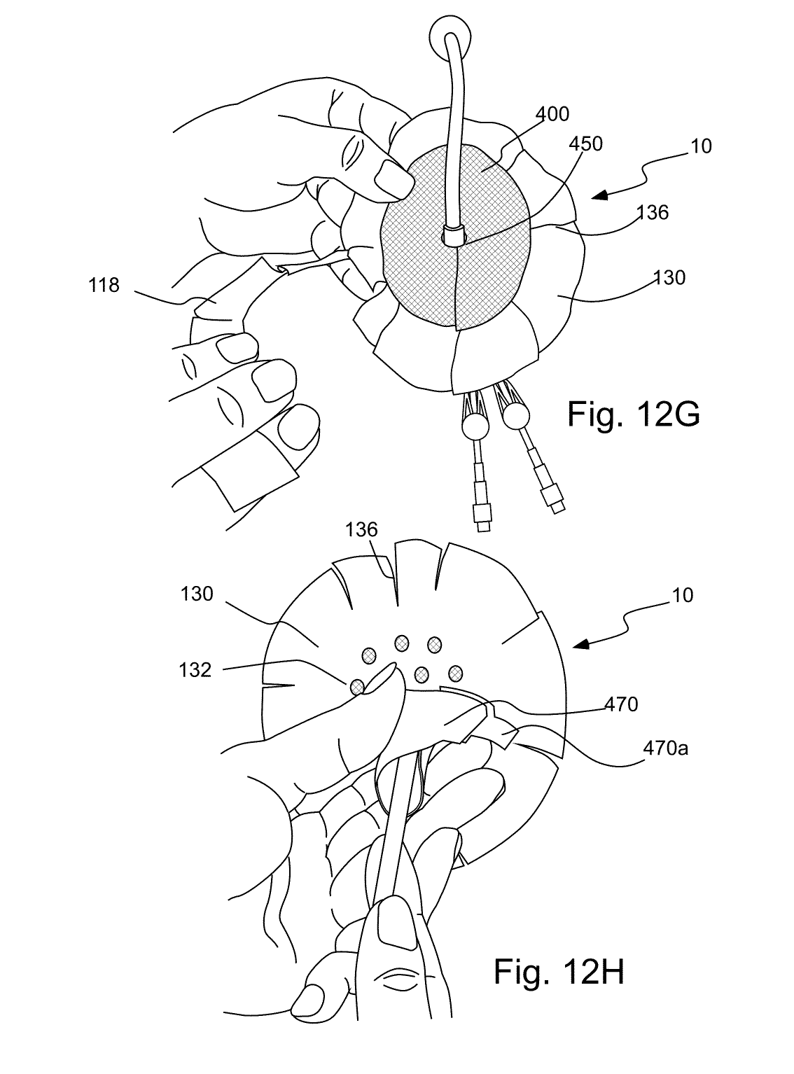

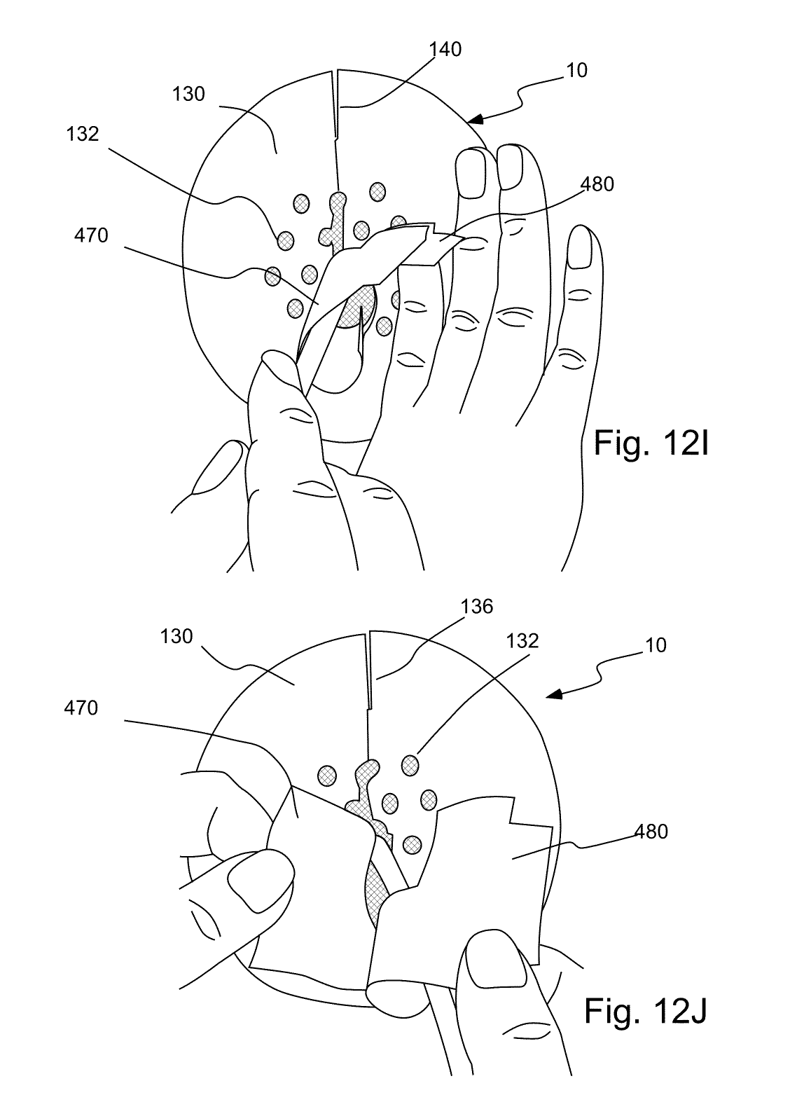

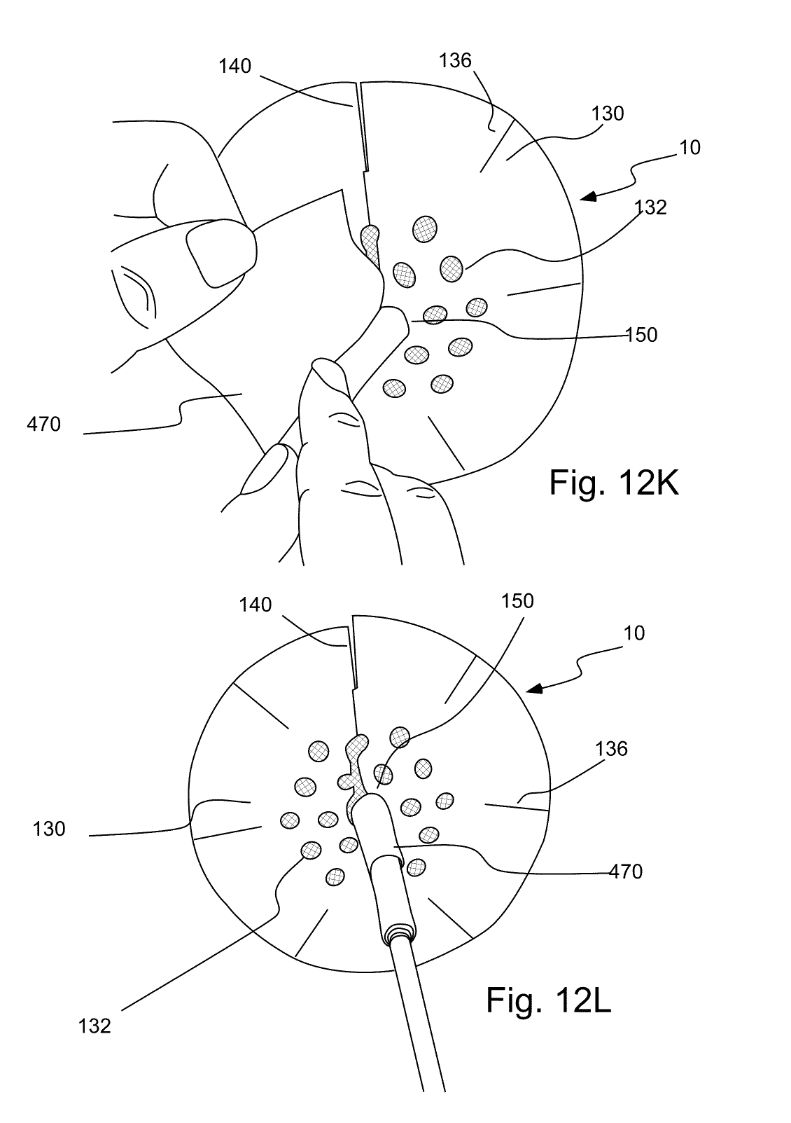

During assembly of port guard dressing 10, second component 200 is adhered to first component 100 by first removing peel away section 112 encompassed within dashed line 122, of FIG. 3, leaving a peel away for a tab 119 with boundaries defined by dashed lines 126 and 141 and section 116, which is formed integrally with tab 119 and is defined by lines 121, a portion of 140, and a portion of 124 (see FIG. 3), thereby exposing adhesive on the bottom surface of outer layer 130 of first component 100 via which an upper surface of second component 200 is adhered to first component 100. In the illustrated embodiment, tab section 119 is a portion of peel away layer 110 connected to section 116 and is made accessible from above upper layer 130 of first component 100 by peeling away tab section 119 and drawing it upward through slit 141 to the position illustrated in FIGS. 1 and 2. Slit 140 provides an opening to generally central passage 150 through which a line, tube, catheter etc. can be passed (see, for example, FIG. 12B). As described further below, when assembled port guard dressing 100 is in place on a patient, the slits in lower components of the illustrated embodiment of port guard dressing 10 are offset from slit 140 by an angle or arc α (see FIG. 2). In other embodiments, slit 140 can be aligned with slits in lower components. In the illustrated embodiments, slit 140 and the slits of the lower components are effectively joined together or closed when tab 119, along with associated/connected peel away layer section 116 are removed (see FIG. 12C) to expose adhesive underlying section 116 (see FIG. 12D) that is used to adhere the portion of upper layer 130 underlying section 116 to second component 200 (see FIG. 12E). FIGS. 12A through 12L illustrate a representative embodiment of sequence of actions for placing a port guard dressing hereof such as port guard dressing 10 in operative connection with a percutaneous line and the skin of a patient surrounding a percutaneous opening through which the percutaneous line passes. In FIGS. 12A through 12L, port guard dressing is shown with only first component 100 and fourth component 400 for simplicity.

The slit and associated generally central passages of each of the components are aligned as illustrated in FIG. 2 during assembly of port guard dressing 10. In that regard, passages 250, 350 and 450 of second component 200, third (intermediate) component 300 and fourth component 400, respectively, are placed in general axially alignment with passage 150. Slits 240, 340 and 440 of second component 200, third component 300 and fourth component 400, respectively, are placed in general alignment with each other and are offset from slit 140 by, for example, 5° to 60° as represented by arc α in FIG. 2. The alignment described above may, for example, be achieved by first assembling the components and the creating all cuts of holes, slits, etc., which entails cutting at the correct depths, after adhering all of the components together. The dashed line defined by 126 and 128 in FIG. 3, however, would still need to be precut. Also, tab section 119 (see FIG. 3), would be required to be peeled away and moved through slit 140 to the other side of 130, as illustrated in FIG. 2.

The lower surface of second component 200, which is opposite the upper surface facing first component 100, may have an adhesive layer and a protective peel away layer (not shown). The peel away layer may be removed to expose adhesive for adhering second component 200 to third component 300, wherein second component 200 and third component 300 may be aligned so that slits 240 and 340 are in general alignment (and offset by angle α with respect to slit 140). Again, the slits may be precut and appropriately aligned during assembly or the slits can be cut after two, more than two or all the layers have been laminated together. In a number of embodiments, layer 300 is an absorbent layer that may, for example, be formed from cotton, polyester, polyurethane, or a mixture thereof. The absorbent material may include hydroscopic powder, gel and/or other material in the matrix thereof. In a number of embodiments third component 300 is formed from a porous, sponge-like material which can absorb fluids (for example, a porous cotton or polyurethane).

Fourth and bottom component 400 (see FIGS. 2 and 6-7B) is placed in direct contact with the skin and percutaneous opening in the skin. Component 400, which is slightly larger in diameter than second component 200 and third component 300 in the illustrated embodiment, may be attached or laminated to first component 100 using an adhesive layer on first component 100 exposed by removing section 114 of peel away layer 110, which is defined by dashed lines (cuts) 122 and 124, as bounded by dashed line 126 and slit 140 (see FIG. 3). The adherence of fourth component 400 to first component 100 effectively sandwiches second component 200 and third component 300 between first component 100 and fourth component 400. Fourth component 400 may, for example, be formed from a layer of fabric such as a cotton fabric, a polymeric fabric, or another textile material. Examples of suitable fabrics include those woven from cotton, polyamide, polyester, an polyacrylate. In a number of embodiments, fourth component 400 was formed from DACRON®, a polyethylene terephthalate (polyester) available from Invista North America S.A.R.L. of Charlotte, North America. The layer of fabric or other material may, for example, be treated with an antibacterial coating. Fourth component 400 completely seals the skin opening from the outside environment in which infection causing micro-organisms can be present. By including an antibacterial coating on component 400 (for example, at least on the bottom surface thereof), fourth component 400 operates as an impenetrable barrier preventing live micro-organisms from passing from the outside environment or surroundings to the percutaneous hole, thereby inhibiting infections. In a number of embodiments, fourth component 400 includes at least one generally flat, radially extending layer of material 430 and an axially extending, tubular member 470 which encompasses a length of a tube or line when port guard dressing 10 is places in operative connection with a patient. Member 470 may, for example, include a slit or opening 472 in general alignment with slit 440 to enable position of the tube or line within member 470.

Fourth component 400 may be constructed in a number of ways. For example, fourth component 400 may be made by weaving or knitting from a single, monolithic piece of material. Fourth component 400b may alternatively be made of fabricated from two (or more) pieces of material 430a and 470a as illustrated in FIG. 7B. As illustrated in FIG. 7B, member 470 may begin as a flat sheet of material 470a with slits 474 cut along bottom edge thereof. Slits 474 create tabs 476 therebetween. When sheet of material 470a is rolled to form extending member 470, tabs 476 may be used to fastened extending member 470 to disk-shaped layer 430 by, for example, sewing, adhesive, and/or ultrasonic welding. During fabrication, slit 472 is aligned with slit 440.

In assembled port guard dressing 10, the area of outer layer 130 between slit 140 and aligned slits 240, 340 and 440 as represented by arc α, provides an openable fold of material in outer layer 130 of upper component 100 (in operative association with aligned slits 240, 340 and 440) via which port guard dressing 10 can be placed in operative connection which a percutaneous line passing through the percutaneous opening in the patient (see, for example, FIG. 12B). After the tube or line is inserted to pass through generally centrally aligned openings 150, 250, 350 and 450, the area between slit 140 and aligned slits 240, 340 and 440 is effectively sealed together by pulling peel away tab 119 to remove section 116 and expose adhesive on first component 100 that overlaps and adheres to second component 200 in an overlapping area thereof to mend the offset slits together and make a stronger bandage that is able to firmly secures the tube or driveline. As illustrated in FIG. 1B, an adhesive or self-adhering strip 480 (for example, a COBAN® strip or wrap available from 3M of St. Paul, Minn.) may be attached to the generally tubular or cylindrical extending member 470 of the fourth, bottom component 400. Adhesive or self-adhering strip 480 may, for example, be attached to extending member 470 at an end thereof and, after attachment of port guard dressing 10 to the patient, then be wrapped around and physically secure the tube, catheter, feeding tube, drain tube, VAD driveline or any other percutaneous line or tube that enters the body via a percutaneous site to the dressing itself. Adhesive or self-adhering strip 480 may, for example, be used to secure a VAD driveline or other percutaneous line. For a more self-contained dressing, adhesive or self-adhering strip 480 may be pre-fastened or adhered to generally tubular or cylindrical extending member 470 as illustrated in FIG. 1B. In the case of an adhesive strip 480, a peel away layer can be used to cover the adhesive before use. The peel away layer may be removed and strip 480 may be wrapped around the tube or line and overlapped after port guard dressing 10 has been placed on the skin and the tube/driveline put in place within extending member 470 (see, for example, FIGS. 12H through 12L).

For attachment of port guard dressing 10 to the skin of the patient, outer section 118 of peel away layer 110 may be removed to expose an underlying adhesive layer or portion that adheres to the skin of the patient (see, for example, FIGS. 12F through 121. In that regard, the bottom surface of outer layer 130 of upper, first component 100, radially outside of the perimeter defined by defined by dashed line 124, includes a skin compatible adhesive thereon that is under section 118 of the peel away layer 110. As previously mentioned, section 118 of peel away layer 110 remains intact after assembly of all components of port guard dressing 10 as shown, for example, in FIG. 2. As also described above, section 116 of peel away layer 110 also remains intact until port guard dressing 10 is to be used in connection with a patient. Once again, the adhesive layer underlying section 116 is used for final securement of the slits as described above. All other section of peel away layer 110 are removed during assembly as described above.

It has been found that a primary reason for the irritation of skin from a dressing adhered to the skin for an extended period of time is the disconnect of elastic properties of the adhered dressing section and the skin. By, for example, placing slits or openings 136 (see, for example, FIGS. 2 and 3) along the circumference of radially outward adhesion section of the port guard dressing 10 (that is, that section of outer layer 130 underlying peel away layer section 118) in contact with the skin, skin irritation is greatly reduced. In that regard, slits or openings 136 allow for independent stretching of port guard dressing 10 and the skin.

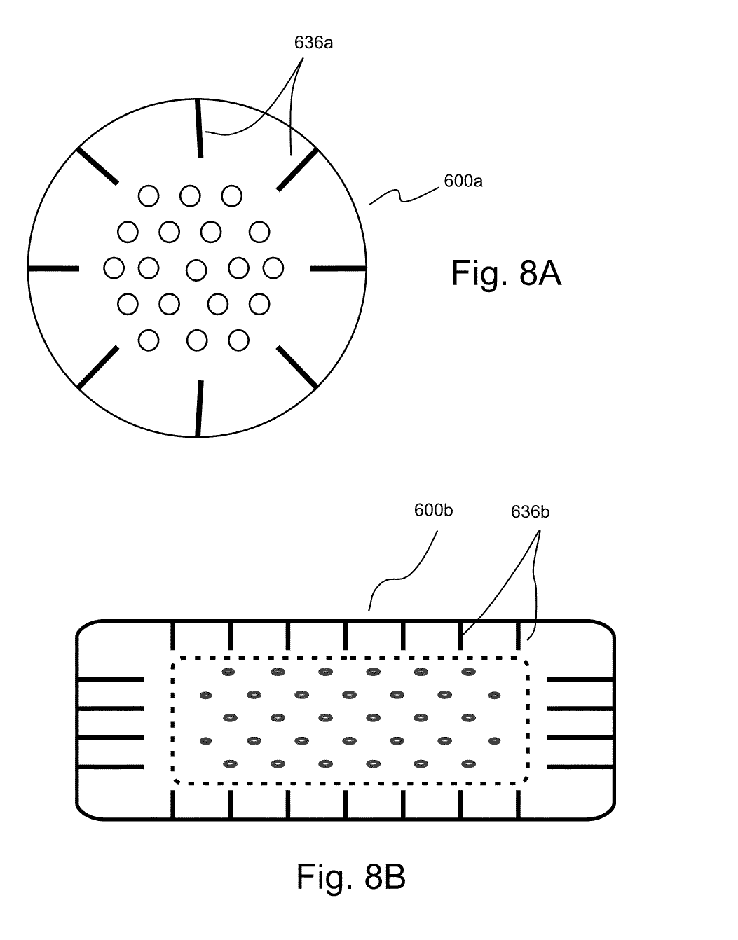

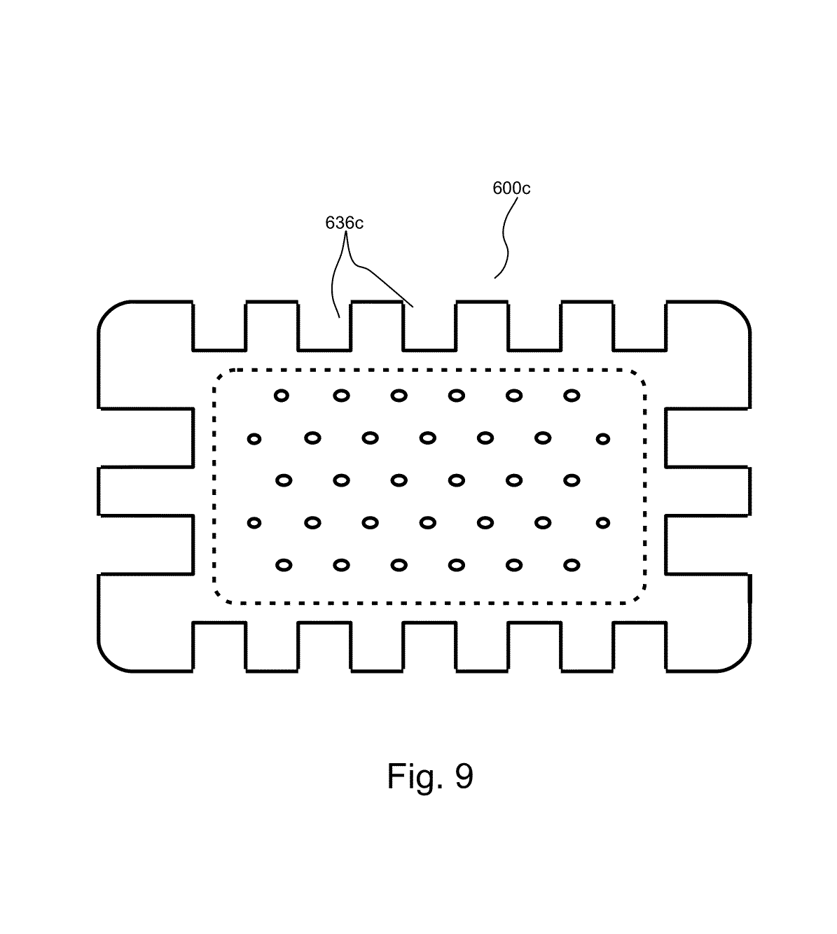

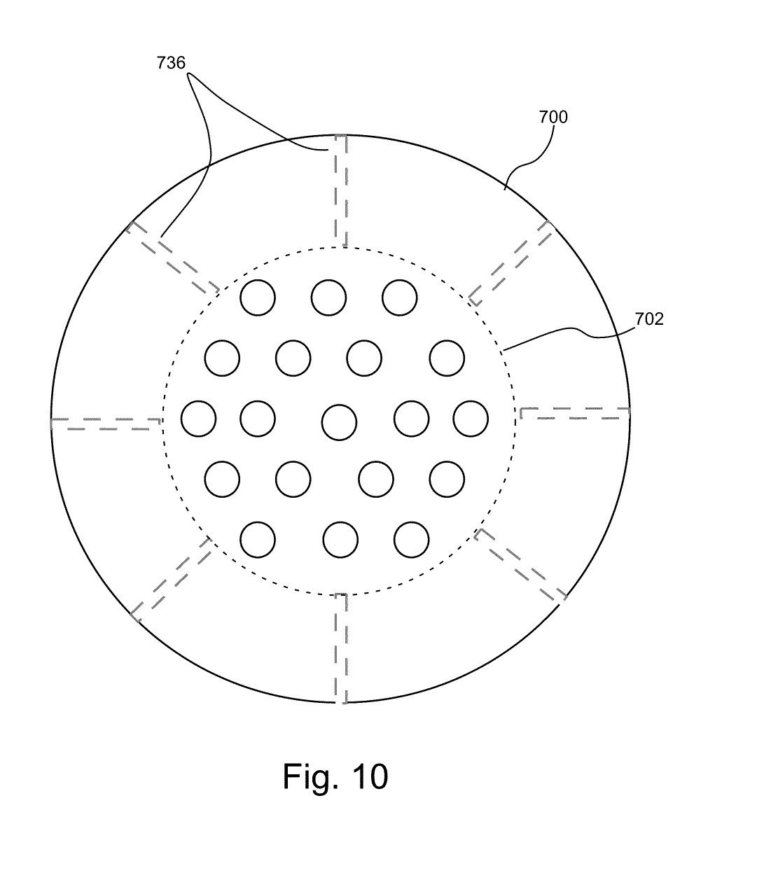

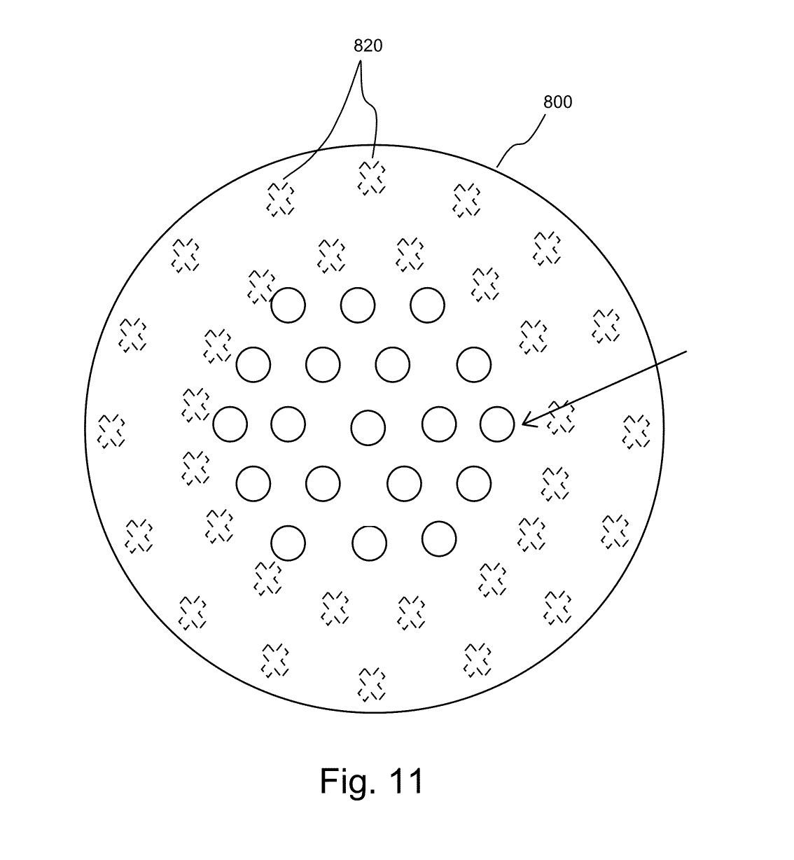

The use of slits or openings for the reduction of skin irritation of bandages or dressing can be used on other bandages or dressings of various shapes and sizes. A couple examples of common bandages 600 and 600a that display the irritation relieving slits or openings 636a and 636b, respectively, are shown in FIGS. 8A and 8B. These irritation relief slits or openings can be broadened to a point where the actual bandage or dressing shape is altered such as shown in FIG. 9 for bandage 600c and openings 636c. In an extension of the above-described irritation relief mechanism, areas or sections of a bottom surface or layer of a bandage or dressing that includes an adhesive layer or layers may be provides with section or portions that do not have adhesive. No slits or cuts need be formed in such a bandage or dressing. In the representative example of bandage 700 of FIG. 10, areas or sections 736 (bounded by dashed lines) of the underside of bandage 700 do not have an adhesive, while the area outside of sections 736 and radially outward of dashed line 702 includes an adhesive layer. The area outside of dashed line 702 may be covered with a peel away layer to be removed when bandage 700 is to be applied to skin. In another embodiment of a skin irritation relief mechanism hereof an adhesive which is not a continuous, uniform adhesive layer (typical for most dressings and other adhered items) is used. Rather, a pattern of adhesive which allows for the disconnect of the skin to dressing is used. FIG. 11 illustrates an example of a bandage or dressing 800 wherein X-shaped areas or sections 820 represent discontinuous or discrete areas where adhesive has been applied. As clear to those skilled in the art, there are numerous other shapes and patterns of adhesive that could be utilized (such as dots, etc.) in various patterns or configurations (such as, checker board layouts, screen patterns, etc.).

In a number of representative embodiments of multi-component/multi-layer port guard dressings 10 hereof, first component 100 may, for example, have a diameter in the range of approximately 1.5 to 5.0 inches. Outer layer 130 of first component 100 may, for example, be fabricated from cloth, elastomeric plastic, nonwoven or other material that is suitable as an upper dressing layer. As described above, second component 200 is more rigid and/or stiffer that first component 100 to provide stiffness and mechanical strength and may, for example, be fabricated from a polymeric material. Second component 200 may, for example, be in the range of approximately 0.05 mm to 1.6 mm inches in thickness and have a diameter in the range of approximately 1 to 3 inches. Absorbent, third component 300 (which may, for example, be formed of cotton, a polymer such as a polyester, or other fabrics or blends of fabrics, whether woven or non-woven) may include a fluid absorbing powder, gel, fibers or other material for absorbing water-based body fluids and/or providing water for activating any anti-microbial coating that may be incorporated in fourth component 400. Third component 300 may, for example, have a diameter that is the same or slightly smaller than second component 200, and smaller than a diameter of bottom, fourth component 400. Fourth component 400 may, for example, have a diameter in the range of approximately 1.0 to 3.25 inches and which is intermediate between the diameters of first component 100 and second component 200. Generally tubular or cylindrical extending member 470 of fourth component 400 may, for example, be approximately 0.5 to 2.0 inches in length and may be formed of the same material or fabric of layer 430 of fourth component 400. As discussed above, extending member 470 may, for example, assist in supporting or holding the tube or line that passes into the body through the percutaneous hole.

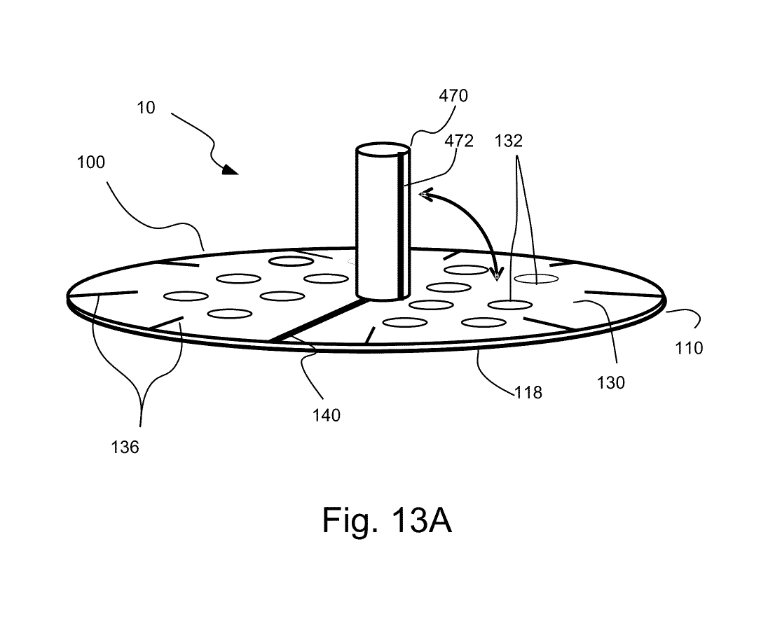

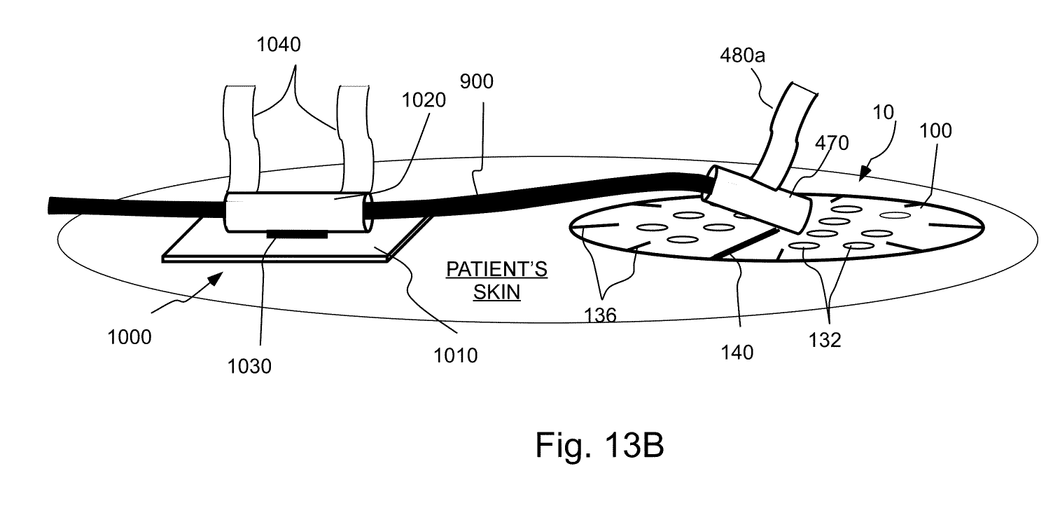

FIG. 13A illustrates another perspective view of the port guard dressing 10 of FIG. 1A demonstrating that member 470, which encompasses a length of a tube or line 900 passing through port guard dressing 10 can be bent to various angles. In FIG. 13B, member 470 port guard dressing and tube or line 900 passing therethrough (for example, a peripherally inserted central catheter (PICC) or other catheter, tube or line) are flexed or bent so that tube or line 900 may be passed through a secondary securement 1000 attached to the patient's skin (for example, through a suitable adhesive as known in the medical arts and described above). In that regard, securement 1000, includes a base member 1010 including, for example, an adhesive coated backing (lower side) for attachment to the patient's skin. A tube or line guide or attachment member 1020 is attached to an opposite (upper) side of base 1010 via, for example, a connector such as a layer of adhesive 1030. Self-adhesive strips 1040 or other types of securing members may, for example, be provided to further secure line guide or attachment member 1020 and/or tube or line 900.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A port guard dressing for use in connection with skin of a patient and through which a percutaneous line can pass, comprising: an upper component, a lower component adapted to contact the skin, and at least one intermediate component positioned between the upper component and the lower component, the at least one intermediate component being attached to a lower surface of the upper component and comprising a material that is more rigid than the upper component.

2. The port guard dressing of claim 1 further comprising at least one absorbent component positioned below the at least one intermediate component and above the lower component.

3. The port guard dressing of claim 1 wherein the upper component comprises a passage through which the percutaneous line can pass and a slit in connection with the passage of the upper component and extending to a perimeter of the upper component, the at least one intermediate component comprises a passage through which a percutaneous line can pass and a slit in connection with the passage of the at least one intermediate component and extending to a perimeter of the at least one intermediate component, and the lower component comprises a passage through which a percutaneous line can pass and a slit in connection with the passage of the lower component and extending to a perimeter of the lower component, the passage of the upper component, the passage of the at least one intermediate component and the passage of the lower component being in general alignment, the slit of the at least one intermediate component and the slit of the lower component being in general alignment and being offset from the slit of the upper component.

4. The port guard dressing of claim 1 wherein the upper component comprises a passage through which the percutaneous line can pass and a slit in connection with the passage of the upper component and extending to a perimeter of the upper component, the at least one intermediate component comprises a passage through which a percutaneous line can pass and a slit in connection with the passage of the at least one intermediate component and extending to a perimeter of the at least one intermediate component, and the lower component comprises a passage through which a percutaneous line can pass and a slit in connection with the passage of the lower component and extending to a perimeter of the lower component, the passage of the upper component, the passage of the at least one intermediate component and the passage of the lower component being in general alignment, the slit of the upper component, the slit of the at least one intermediate component, the slit of the lower component being in general alignment.

5. The port guard dressing of claim 3 further comprising at least one absorbent component positioned below the at least one intermediate component and above the lower component, the absorbent component comprising a passage through which a percutaneous line can pass and a slit in connection with the passage of the absorbent component and extending to a perimeter of the absorbent component, the passage of the absorbent component being in general alignment with the passage of the upper component, the passage of the at least one intermediate component and the passage of the lower component, the slit of the absorbent component being in general alignment with the slit of the at least one intermediate component and the slit of the lower component.

6. The port guard dressing of claim 3 wherein the upper component comprises a first section comprising an adhesive on a lower surface thereof adjacent the slit of the upper component via which the upper component is adapted to be adhered to at least one of the intermediate component and the lower component after the percutaneous line is positioned to pass through the passage of the upper component, the passage of the at least one intermediate component and the passage of the lower component via the slit of the at least one intermediate component and the slit of the lower component.

7. The port guard dressing of claim 6 wherein the first section is covered with a first section of a removable layer.

8. The port guard dressing of claim 7 wherein the upper component is adhered to at least one of the intermediate component and the lower component via an adhesive positioned on a lower surface of the upper component outside of the first section of the upper component during fabrication of the port guard dressing.

9. The port guard dressing of claim 8 wherein an area of the upper component is larger than an area of the at least one intermediate component and an area of the lower component and the upper component comprises a second section comprising an adhesive on the lower surface thereof via which the port guard dressing is adapted to be attached to the skin of the patient, the second section extending around a perimeter of the upper component.

10. The port guard dressing of claim 9 wherein the second section of the upper component comprises a plurality of openings formed therein which are adapted to decrease skin irritation, wherein the plurality of openings extend from a perimeter of the second section inward toward the passage of the upper component.

11. The port guard dressing of claim 1 wherein the upper component comprises a plurality of holes therein and the at least one intermediate component comprises a plurality of holes therein, the plurality of holes of the upper component being generally aligned with the plurality of holes of the at least one intermediate component.

12. The port guard dressing of claim 1 wherein the at least one intermediate component comprises an adhesive layer on a lower surface thereof.

13. The port guard dressing of claim 1 wherein the lower component comprises a layer of material adapted to contact the skin and a generally axially oriented cylindrical extending member which extends from the layer of material of the lower component through the passage of the at least one intermediate component and the passage of the upper component, the extending member comprising an axially extending slit therein in general alignment with the slit of the at least one intermediate component and the slit of the lower component through which the percutaneous line can pass to be placed within the extending member.

14. The port guard dressing of claim 13 further comprising a strip of material which is adapted to be wrapped around the extending member and the percutaneous line.

15. The port guard dressing of claim 14 wherein the strip of material comprises an adhesive or is self-adhering and is attached to the extending member at one end thereof.

16. A port guard dressing for use in connection with skin of a patient and through which a percutaneous line can pass, comprising: an upper component, a lower component adapted to contact the skin, and at least one intermediate component positioned between the upper component and the lower component, the upper component comprising a passage through which the percutaneous line can pass and a slit in connection with the passage of the upper component and extending to a perimeter of the upper component, the at least one intermediate component comprising a passage through which a percutaneous line can pass and a slit in connection with the passage of the at least one intermediate component and extending to a perimeter of the at least one intermediate component, and the lower component comprising a passage through which a percutaneous line can pass and a slit in connection with the passage of the lower component and extending to a perimeter of the lower component, the passage of the upper component, the passage of the at least one intermediate component and the passage of the lower component being in general alignment, the slit of the at least one intermediate component and the slit of the lower component being in general alignment, and the slit of the upper component being offset from the slit of the at least one intermediate component and the slit of the lower component.

17. The port guard dressing of claim 17 wherein the upper component comprises a first section comprising an adhesive on a lower surface thereof adjacent the slit of the upper component via which the upper component is adapted to be adhered to at least one of the intermediate component and the lower component after the percutaneous line is positioned to pass through the passage of the upper component, the passage of the at least one intermediate component and the passage of the lower component via the slit of the at least one intermediate component and the slit of the lower component.

18. A method of fabricating a port guard dressing for use in connection with skin of a patient and through which a percutaneous line can pass, comprising: providing an upper component, providing a lower component adapted to contact the skin, providing at least one intermediate component positioned between the upper component and the lower component, attaching the at least one intermediate component to a lower surface of the upper component, the at least one intermediate component comprising a material that is more rigid than the upper component.

19. A method of fabricating a port guard dressing for use in connection with skin of a patient and through which a percutaneous line can pass, comprising: providing an upper component, providing a lower component adapted to contact the skin, providing at least one intermediate component positioned between the upper component and the lower component, the upper component comprising a passage through which the percutaneous line can pass and a slit in connection with the passage of the upper component and extending to a perimeter of the upper component, the at least one intermediate component comprising a passage through which a percutaneous line can pass and a slit in connection with the passage of the at least one intermediate component and extending to a perimeter of the at least one intermediate component, and the lower component comprising a passage through which a percutaneous line can pass and a slit in connection with the passage of the lower component and extending to a perimeter of the lower component, the passage of the upper component, the passage of the at least one intermediate component and the passage of the lower component being in general alignment, the slit of the at least one intermediate component and the slit of the lower component being in general alignment, and the slit of the upper component being offset from the slit of the of the at least one intermediate component and the slit of the lower component.
